(19) United States
(12) Patent Application Publication
(10) Pub. No.: US 2022/0332798 A1
(43) Pub. Date: Oct. 20, 2022

(54) PRODUCTION OF ANTI-VIRAL MONOCLONAL ANTIBODIES BY HYDRODYNAMIC-BASED TRANSFECTION

(71) Applicant: Oregon Health & Science University, Portland, OR (US)

(72) Inventors: Hiroyuki Nakai, West Linn, OR (US); Kei Adachi, Beaverton, OR (US); Helen Rappe Baggett, Hillsboro, OR (US)

(21) Appl. No.: 17/765,394

(22) PCT Filed: Oct. 2, 2020
(86) PCT No.: PCT/US20/54151

§ 371 Date: Mar. 30, 2022

Related U.S. Application Data
(60) Provisional application No. 62/909,650, filed on Oct. 2, 2019.

(51) Int. Cl. C07K 16/08

(52) U.S. Cl. CPC C07K 16/081

(57) ABSTRACT

The present disclosure relates to methods of producing monoclonal antibodies in animals. In particular, the disclosure provides a method of producing, in vivo, antibodies against viral capsids (VCs) derived from a non-enveloped virus (NEV). The method includes administering to a subject, by hydrodynamic-based transfection, a first set of genetic material encoding NEV structural proteins to induce the subject's intracellular translation and assembly of the proteins into viral capsids. The method also includes administering a second set of genetic material encoding NEV non-structural proteins to facilitate the intracellular assembly of the NEV structural proteins. Thus, this method may be used to produce subject-generated antibody-producing cells that secrete anti-VC antibodies that may be harvested and screened for monoclonal anti-VC antibodies.

### STATEMENT REGARDING FEDERALLY SPONSORED RESEARCH

This invention was made with government support under R01 NS088399 awarded by the National Institutes of Health. The government has certain rights in the invention.

### COPYRIGHT NOTICE

©2020 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

Generally, the field relates to methods for producing antibodies against viral capsids. More specifically, the field relates to methods for producing monoclonal antibodies against adeno-associated virus (AAV), viruses in the family Parvoviridae, and other non-enveloped viruses by hydrodynamic-based transfection.

### BACKGROUND INFORMATION

Monoclonal antibodies can serve as powerful scientific and therapeutic tools. However, effective animal immunization against an antigen, giving rise to the production of monoclonal antibodies, can be a very complicated, time-consuming, and expensive process. Generation of the antigen itself can also be similarly difficult process. Moreover, to be effective, traditional immunization methods often require that additional peptides, proteins or other adjuvants be added to the inoculum, risking side effects to the animal subject. And selecting an antibody with enough specificity to be deemed “monoclonal” requires the screening of large libraries of parent cells generated by the immunization. Thus, an immunization process that generates parent cells without the complexity, time, and expense of traditional methods facilitates the production of monoclonal antibodies, allowing for greater access to their scientific and therapeutic benefits.

### SUMMARY OF THE DISCLOSURE

The current disclosure provides a method to produce, in vivo, antibodies against viral capsids (VCs) that derive from a non-enveloped virus (NEV). Some of the embodiments are configured to antibodies against virus-like particles (VLPs). Step 1 of a preferred method administers, to a subject, by hydrodynamic-based transfection, a first set of genetic material encoding one or more NEV structural proteins that induces the subject's intracellular translation and assembly of the NEV proteins into one or more VCs. The subject, thereby, generates antibody-producing cells that secrete anti-VC antibodies. Step 2 of the preferred method includes optionally repeating step 1.

In addition, the disclosure provides a method containing the step of administering to the subject, by hydrodynamic-based transfection, a second set of genetic material encoding one or more NEV non-structural proteins for facilitating the subject's intracellular assembly of the NEV structural proteins into one or more VCs.

In addition, the disclosure provides a method containing the step of harvesting antibody-producing cells and/or anti-VC antibodies from the subject.

In addition, the disclosure provides a method containing the step of generating a plurality of hybridomas from the antibody-producing cells. The plurality of hybridomas is screened and one or more hybridomas from within the plurality is selected for culture and one or more of the selected hybridomas is cultured.

In some embodiments, step 1 of the method is repeated 1 to 10 times. In other embodiments, the time intervals between repeating step 1 are about 1 day to about 28 days.

In some embodiments, the first and second set of genetic material are at least one nucleic acid molecule chosen from a ssDNA, a dsDNA, a linear pDNA, a circular pDNA, a ssRNA, a dsRNA, a circRNA, a mRNA, an siRNA, a microRNA, and a sgRNA. In other embodiments, the first set of genetic material encodes at least one protein chosen from a NEV-based VP capsid protein, an AAV VP capsid protein, an AAV2 VP capsid protein, and an AAVAnc80 VP capsid protein. In further embodiments, the second set of genetic material encodes at least one protein chosen from NEV-based non-structural proteins that facilitate capsid assembly, an AAV-based AAP, an AAV2 AAP, and an AAPAnc80. In other embodiments, either the first or the second set of genetic material encode a gene expression cassette chosen from an enhancer, a promoter, or a polyadenylation signal.

In some embodiments, the non-enveloped virus is classified within the family Parvoviridae, or is an adeno-associated virus (AAV), a synthetic AAV, or an AAV vector. In other embodiments, the AAV vector is an AAVAnc80 vector, an AAV2 vector, or an AAV9 vector.

In some embodiments, the hydrodynamic-based transfection is performed substantially free of vaccine additive.

In some embodiments the subject is an animal. In other embodiments, the animal is a rodent. In further embodiments, the rodent is a mouse.

In some embodiments, anti-VC antibodies substantially neutralize a NEV's infectivity. In other embodiments, anti-VC antibodies do not substantially neutralize a NEV's infectivity. In further embodiments, the VCs constitute one or more substantially assembled, or partially assembled, NEV viral capsids. In still further embodiments, the anti-VC antibodies are mouse monoclonal antibodies.

Additional aspects and advantages will be apparent from the following detailed description of preferred embodiments, which proceeds with reference to the accompanying drawings.

### BRIEF DESCRIPTION OF THE DRAWINGS

FIGS. 1A and 1B are bar graphs representing, respectively, AAV2VP3 virus-like particle (VLP) production in mouse liver following hepatocyte in vivo transfection with plasmid DNA and the presence of VLP subsequently secreted into the blood. FIG. 1A shows the amount of VLPs per 100 mg liver tissue where a naive animal served as a control. FIG. 1B shows the VLP concentration in the blood. Fifty μg each of pCMV-AAV2VP3 and pCMV-AAP2 plasmids were hydrodynamically injected into three C57BL/6J male mice. Three days post-injection, the blood and liver samples were subjected to titration of AAV2VP3 VLP particles by an AAV2 Titration ELISA (PROGEN).

FIG. 2 is a bar graph showing AAV VP3-AAP cross-complementation. HEK293 cells were seeded onto 6-well plates 1 day before transfection. Cells were then transfected with pEMBL-CMV-GFP, pCMV-AAVxVP3, pCMV-AAPy, pHLP-Rep, and pHELPER as previously described (Earley, Kawano et al. 2015, Earley, Powers et al. 2017). “x” and “y” in pCMV-AAVxVP3 and pCMV-AAPy represent AAV serotypes or variants (x=2, 9 and Anc80 and y=1, 2, 3, 4, 5, 6, 7, 8, 9, 10, 11 and 12, respectively). pEMBL-CMV-GFP is an AAV vector plasmid for the production of double-stranded (ds) AAV-CMV-GFP vector. As shown in FIG. 2, the x-axis and y-axis represent, respectively, the AAV serotypes or variants and the yield of AAV VP3 in the 6-well plates. At 5 days post-transfection, the medium and cells were harvested and dsAAV-CMV-GFP AAVxVP3 vector yields were determined by a quantitative dot blot analysis (Powers, Chang et al. 2018). In addition, the y-axis shows AAV VP3 only viral particle concentration in culture media,

FIGS. 3A and 3B are, respectively, a bar graph and a box plot representing titers of serum antibodies against assembled AAV2VP3, AAV9VP3 and AAVAnc80VP3 viral capsids in mice immunized by hydrodynamic tail vein injection of plasmid DNA. Anti-AAV capsid antibody titers were determined by anti-AAV capsid antibody ELISA. As shown in FIG. 3A, BALB/cJ female mice were immunized by hydrodynamic tail vein injection of pCMV-AAVxVP3 plasmid together with AAPy plasmid using the method described above (x=Anc80, 2, and 9, and y=2, 2, and 9, respectively). Mice ID 1 to 10 were for AAVAnc80VP3 capsid immunization, Mice ID 14 and 15 were for AAV2VP3 capsid immunization, and Mice ID 16 and 17 were for AAV9VP3 capsid immunization. Mice ID 11, 12 and 13 were untreated. Serum samples from each mouse were assayed by anti-AAV capsid antibody ELISA for the presence of anti-AAVAnc80 capsid antibodies (black bars), anti-AAV2 capsid antibodies (gray bars) and anti-AAV9 capsid antibodies (white bars). All the immunized mice produced corresponding anti-AAV capsid antibodies. Anti-AAV2 antibodies produced in mice immunized with AAV2VP3 plasmid were found to cross-react with AAVAnc80VP3 capsids. Similarly, anti-AAV9 capsid antibodies produced in mice immunized with AAV9VP3 plasmid were found to cross-react with AAVAnc80VP3 capsids but to a lesser degree. FIG. 3B represents anti-AAVAnc80 capsid antibody titers in sera collected from BALB/cJ female mice immunized with AAVAnc80VP3 plasmid DNA injection (n=10) and the samples collected from untreated naive mice (n=3) (as shown in FIG. 3A). The amounts of antibodies against AAV2VP3 capsids, AAV9VP3 capsids, and AAVAnc80VP3 capsids determined by ELISA are presented as OD490 values in a box plot. A Welch's t-test was used for statistical comparison of the data between the two groups.

FIG. 4 is a bar graph representing the residual viral infectivity after injecting mice with plasmid DNA three times in a period of 3 weeks and five times in a period of 5 weeks for production of anti-AAVAnc80 neutralizing antibodies (NAbs). Relative light unit (RLU) values obtained from the NAb assay at a 1:1,000 serum dilution are shown only for Mice 4, 7 and 8 (See Table 2, disclosed herein). As shown in FIG. 4, there was enhancement of NAb-mediated inhibition of HEK293 cell transduction when the 5-week time point samples were assayed at this dilution, demonstrating that five times injection enhances anti-AAVAnc80 NAb titers compared to three times injection.

FIG. 5 is a bar graph representing the neutralizing ability of 19 anti-AAVAnc80 capsid mouse monoclonal antibodies identified by an anti-AAVAnc80-specific antibody ELISA. From the monoclonal antibodies identified from two BALB/cJ female mice immunized with AAVAnc80VP3 capsids by the methods disclosed herein, 19 monoclonal antibodies were selected for a neutralizing antibody (NAb) assay using AAVAnc80-CMV-luciferase (AAVAnc80-CMV-luc) vector and HEK293 cells as a reporter cell line. As shown in FIG. 5, the y-axis indicates relative transduction efficiency of AAVAnc80-CMV-luc vector (%) in the presence of each monoclonal antibody (moAb)-containing hybridoma culture supernatant compared to the transduction efficiency obtained from the reference control in which AAVAnc80-CMV-luc vector transduced HEK293 cells in the absence of antibodies. Among the 19 moAbs, 6 were neutralizing antibodies and 13 were non-neutralizing antibodies. MoAbs with one or two asterisks were characterized in detail. MoAbs with two asterisks were selected for purification by affinity chromatography.

FIG. 6 is a bar graph representing the cross-reactivity of anti-AAVAnc80 capsid monoclonal antibodies (moAbs) to other serotypes. Cross-reactivity of anti-AAVAnc80 capsid moAbs, 1B11, 2A7, 5B10, 4B7, 3D6, 2F11, and 1A11 were assessed by anti-AAVx capsid antibody ELISAs (x=AAVAnc80, AAV2, AAV5 and AAV9). Affinity-purified moAbs were used for the assay except for 1A11, which was hybridoma supernatant. Y-axis indicates OD490 values in the ELISAs. MoAbs 1B11, 2A7, 5B10, 4B7, and 3D6 displayed no cross-reactivity to AAV2, AAV5, and AAV9. MoAb 5B10 showed weak binding to AAVAnc80. MoAb 2F11 showed cross-reactivity to AAV2. MoAb 1A11, which binds to AAVAnc80 capsid weakly, exhibited weak but broad cross-reactivity to other serotypes. Values are mean±SD (n=2).

FIG. 7 is a bar graph representing the effective neutralization of AAVAnc80-CMV-luc vector infectivity by affinity-purified anti-AAVAnc80 capsid mouse monoclonal antibodies (moAbs), 2A7 and 1B11. Affinity-purified moAbs with concentrations of 2.6 mg/mL and 3.1 mg/mL were diluted by the dilution factors indicated in the figure (1:1,000, 1:10,000, 1:100,000, and 1:1,000,000). Ten μL of AAVAnc80-CMV-luc vector preparation containing 10⁹ particles were reacted with 10 μL of the diluted moAb and subjected to the neutralizing antibody (NAb) assay using CHO-K1 cells as a reporter cell line. Transduction efficiencies with the vector were determined by luciferase expression and compared to the values obtained in the no-moAb control condition under which the assay was performed in the absence of NAbs. NAb titer is commonly defined as the highest dilution that suppresses the relative transduction by >50%. With this definition, the affinity-purified moAbs 2A7 and 1B11 had NAb titers of >1:10,000.

FIGS. 8A, and 8B represent the detection limits of anti-AAVAnc80 capsid ELISAs with 2A7 and 1B11 mouse monoclonal antibodies (moAbs), and their specificities to AAVAnc80 capsids, respectively. Ninety-six-well ELISA plates were coated with 100 ng of 2A7 anti-AAVAnc80 capsid moAb (as shown in FIG. 8A) or 1B11 anti-AAVAnc80 capsid moAb (as shown in FIG. 8B). Known amounts of AAVAnc80, AAV2, AAV5, and AAV9 viral particles as indicated in the figure were added to each well. Fifty ng of biotinylated 2A7 or 1B11 was added to the corresponding wells, and the antigen-bound biotinylated moAbs were detected by streptavidin conjugated with horseradish peroxidase (Streptavidin-HRP). OD was measured at 490 nm. The detection limit of anti-AAVAnc80 capsids was 1×10⁶ particles in 50 μL per well in both of the 2A7-based and 1B11-based AAVAnc80 capsid ELISAs. OD490 values for the background, 10⁶, and 10⁷ were 0.051, 0.074, and 0.240, respectively, for the 2A7 moAb. OD490 values for the background, 10⁶, and 10⁷ were 0.047, 0.081, and 0.316, respectively, for the 1B11 moAb. Increasing amounts of AAV particles present in wells did not result in an increase of non-specific binding of the moAbs to AAV capsids other than AAVAnc80.

FIGS. 9A and 9B represent standard curves of an AAVAnc80 capsid-specific titration ELISAs for AAVAnc80 particle quantification based on 2A7 moAb and 1B11 moAb, respectively. Ninety-six-well ELISA plates were coated with 100 ng moAb (2A7 or 1B11, as shown). Purified AAVAnc80-CMV-GFP vector was added to the wells in a two-fold dilution series in a range between 7.8×10⁵ and 1.0×10⁸ vg in 50 μL per well. A sandwich ELISA was performed with addition of biotinylated 2A7 or 1B11 moAb. ELISA signals were detected with HRP-conjugated streptavidin and o-phenylenediamine dihydrochloride (OPD). ELISA OD490 values for each condition were collected in triplicate. Standard curves were drawn on a log/log plot of AAVAnc80 capsid concentrations and OD490 values. Pearson's correlation coefficient (r) is given to each panel. The AAVAnc80 capsid-specific titration ELISAs established in this design show a 2-log linear dynamic range with a high correlation coefficient.

### DETAILED DESCRIPTION OF PREFERRED EMBODIMENTS

Disclosed are novel DNA vaccine-based methods to immunize mice effectively without adjuvants. This method can be used for mouse monoclonal antibody production against assembled AAV viral capsids, and more broadly for any parvoviral or non-enveloped capsids as long as capsids can assemble in the mouse body by delivering genetic materials as nucleic acids coding viral components into the body. By delivering such genetic materials into hepatocytes hydrodynamically by tail vein injection in mice, a total of 19 hybridoma clones were identified that produced mouse monoclonal antibodies that recognized assembled capsids. Among the 19 monoclonal antibodies, 6 were found to possess the ability to neutralize AAV infection while the other 13 were found to bind assembled capsid but did not neutralize viral infectivity. All of the 6 neutralizing antibodies and 8 out of the 13 non-neutralizing monoclonal antibodies recognized only assembled capsids and did not bind capsid monomers while 5 of the 13 non-neutralizing monoclonal antibodies could recognize both assembled capsids and capsid monomers. Six monoclonal antibodies were affinity purified for downstream use for various purposes including ELISA, immunoblot, immunofluorescence microscopy, and so on. The sites of plasmid DNA delivery and subsequent viral capsid assembly could be organs and cell types other than the liver, such as skeletal muscles. The method reported here is extremely cost-effective and therefore could become a standard method for production of monoclonal antibodies against viruses in the family Parvoviridae including AAV and other non-enveloped viruses. In addition, the new reagents described herein could be used to create AAVAnc80-specific capsid titration ELISA kits, which do not currently exist.

The present disclosure provides novel methods to generate mouse monoclonal antibodies against assembled capsids of viruses in the family Parvoviridae that are neutralizing or non-neutralizing, by hydrodynamic-based transfection. “Hydrodynamic-based transfection,” as defined herein, is the process of introducing exogenous genetic materials into a subject by hydrodynamic delivery to enhance transfection of the genetic material and thereby facilitate intracellular transgene expression (Liu F. et al., 1999). In some embodiments, the hydrodynamic-based transfection results in the immunization of the subject as a consequence of the subject's intracellular transgene expression of the exogenous genetic material. “Hydrodynamic delivery”, as defined herein, is the application of controlled hydrodynamic pressure in capillaries to enhance endothelial and parenchymal cell permeability. (Suda T. et al., 2007). For example, hydrodynamic delivery includes the pressurized injection of a large volume of solution into a vasculature, such as by hydrodynamic tail vein injection in rodents. “Transfection,” as defined herein, is the process of introducing exogenous genetic material into eukaryotic cells. “Genetic material,” as defined herein, are nucleic acid molecules, examples of which include single-stranded DNA (ssDNA), double-stranded DNA (dsDNA), plasmid DNA (pDNA), linear pDNA, circular pDNA, single stranded RNA (ssRNA), double stranded RNA (dsRNA), circular RNA (circRNA), messenger RNA (mRNA), microRNA, small interfering RNA (siRNA), and single guide RNA (sgRNA).

In one embodiment, the hydrodynamic-based transfection relied on intravenous injection of plasmid DNAs expressing parvovirus structural proteins and non-structural viral proteins that are necessary and sufficient for viral capsid assembly in the body of a mouse, specifically in mouse liver and mouse hepatocytes in vivo. These methods do not require immunization of mice with viral particles or use of any adjuvant or proteins, which significantly simplifies the overall procedure of mouse monoclonal antibody production against assembled parvovirus capsids. Although AAV2 and AAVAnc80 were used for a proof-of-concept study, this disclosure provides methods that can be applied broadly to viruses in the family of Parvoviridae and other types of non-enveloped viruses that can be produced as virus-like particles (VLPs) in the body of a mouse, in mouse liver, and specifically in mouse hepatocytes, in vivo, by delivering genetic materials into the body of mice in vivo that code viral proteins. Although plasmid DNA was used for reduction purposes, genetic materials could be any forms of nucleic acids, including DNA and RNA. The method may also be used to produce mouse monoclonal antibodies against other non-enveloped viruses, and organs and cell types other than the liver and hepatocytes could be targeted for delivery of genetic materials to immunize mice and produce anti-viral capsid antibodies.

The present disclosure also provides antibodies that recognize assembled parvovirus capsids. For example, described herein are 19 novel mouse monoclonal antibodies, 1B11, 1C8, 2A7, 2F11, 3D6, 5B10, 1F10, 2B5, 2D9, 4A12, 4C2, 4D12, 4H10, 6E11, 1A11, 2E3, 4B7, 6F12, and 7F11, that were raised against assembled AAVAnc80 viral capsids and were produced by the method described above. The AAV capsids are composed of 60 subunits comprising VP1, VP2 and VP3 proteins at an approximately 1:1:10 stoichiometric ratio. The fully assembled capsids can be produced only with VP3 protein without packaging viral genomes, making VP3-only empty capsids, namely, virus-like particles (VLPs). VLPs have been widely used for immunizing animals and humans for the purpose of vaccination against infectious diseases and for the purpose of the production of monoclonal and polyclonal anti-viral neutralizing antibodies used as experimental reagents. It was discovered that AAV VP3-only particles (VLPs) can be produced in mouse hepatocytes and can be secreted in the blood circulation when hepatocytes are transfected with two plasmids, one expressing the VP3 protein and the other expressing the assembly-activating protein (AAP) under the control of the cytomegalovirus immediate early gene (CMV-IE) enhancer-promoter. The CMV-IE enhancer-promoter was selected because it is a widely-used ubiquitous and “strong” promoter; however, other enhancer-promoters can also be used for the purpose of immunization. Based on the discovery that AAV4, AAV5 and AAV11 capsid assembly does not require AAP proteins (Earley et al. 2017), it is presumed that VLPs of these serotypes can be produced by transfection of only one plasmid expressing the VP3 protein. It was also discovered that significant immune responses against assembled AAV capsids can be mounted by tail vein injection of the above-described plasmid DNAs without using any additional treatment such as administration of an adjuvant. Based on this discovery, a design was conceived of generating mouse monoclonal antibodies against assembled AAV viral capsids (and potentially any viruses in the family of Parvoviridae and more broadly any non-enveloped viral capsids) by hydrodynamic-based transfection of mice, which can simplify and streamline the procedure of producing mouse monoclonal anti-virus antibodies as well as reduce costs. Here, hydrodynamic-based transfection was employed to produce mouse monoclonal antibodies against AAVAnc80 capsids. As a result, the above-described 19 novel mouse monoclonal antibodies were identified to bind to assembled capsids. Among the 19 monoclonal antibodies, 6 were found to possess the ability to neutralize AAV infection. The other 13 were found to bind to assembled capsid but did not neutralize viral infectivity. All of the 6 neutralizing antibodies, and 8 out of the 13 non-neutralizing monoclonal antibodies, recognized assembled capsids only and did not bind to capsid monomers, while 5 of the 13 non-neutralizing monoclonal antibodies recognized both assembled capsids and capsid monomers. Isotypes have been determined for the following 9 monoclonal antibodies using Mouse Immunoglobulin Isotyping ELISA kit (BD Biosciences): 1B11 (IgG1-κ), 1C8 (IgG3-κ), 2A7 (IgG1-κ), 2F11 (IgG1-κ), 3D6 (IgG1-κ), 5B10 (IgA-κ), 1A11 (IgM-κ), 4B7 (IgG2a-κ), and 6F12 (IgM-κ). The following 6 monoclonal antibodies were purified by affinity chromatography using Thermo Scientific™ Pierce™ Protein G Agarose: 1B11, 2A7, 2F11, 3D6 and 5B10, which can react only with assembled AAVAnc80 capsids, do not recognize capsid monomers and have the ability to neutralize viral infectivity; and 4B7, which reacts with both assembled AAVAnc80 capsids and capsid monomers and is devoid of the ability to neutralize viral infectivity. Out of the 6 monoclonal antibodies, all but 2F11 did not cross-react with AAV2, AAV5 or AAV9 capsids. 2F11 showed cross-reactivity with AAV2 but did not bind to AAV5 or AAV9.

In certain embodiments, the present disclosure provides methods of producing antibodies against assembled viral capsid of a non-enveloped virus comprising the steps of (1) immunizing an animal with a first set of one or more genetic materials encoding one or more viral structural proteins for VLP formation and, when necessary or useful for VLP formation, a second set of genetic materials encoding one or more viral non-structural proteins that function in VLP formation and (2) harvesting antibodies and/or antibody-producing cells from the animal. A “set of genetic materials” may comprise one or more such sequences. In some embodiments, the animals are immunized by tail vein injection. In other embodiments, methods such as the use of lipid nanoparticles deliver the genetic materials to the animal's liver without using high pressure injection. In other embodiments, genetic materials are delivered to an animal's organ other than the liver such as skeletal muscle with high pressure injection or with methods such as the use of lipid nanoparticles. Methods of harvesting antibodies and antibody-producing cells from immunized animals are known in the art. The animal may be a rodent or other suitable animal. In certain embodiments, the animal is a mouse.

In certain embodiments, the antibodies produced by the methods described herein are monoclonal antibodies. Such methods may further comprise the steps of (3) generating a plurality of hybridomas from the antibody-producing cells harvested from the animal, (4) screening the plurality of hybridomas to determine which hybridomas produce antibodies that are able to bind to the fully-assembled viral capsid, and (5) culturing one or more hybridomas that are able to bind to the assembled viral capsid. Methods of producing and culturing hybridomas are known in the art.

In certain embodiments, the non-enveloped virus is a virus in the family Parvoviridae. In some embodiments, the non-enveloped virus is AAV. In particular embodiments, the AAV may be a primate serotype AAV vector. In some embodiments, a primate AAV may be derived from any known serotype, e.g., from AAV1, AAV2, AAV3, AAV4, AAV5, AAV6, AAV7, AAV8, AAV9, AAV10, AAV11, AAV12, AAV13, AAVhu37, AAVrh8, AAVrh10 and AAVrh74. In some embodiments, the non-enveloped virus is a synthetic AAV vector, for example, AAV-DJ, AAV-LK03, AAV-KP1 and AAVAnc80. In some embodiments, the non-enveloped virus is AAV and the second set of nucleic acid sequences encode one or more viral assembly activating proteins (AAPs).

In certain embodiments, at least one of the genetic materials used in the immunization are DNA molecules. In certain embodiments, at least one of the genetic materials are RNA molecules. In certain embodiments, the genetic materials encode a gene expression cassette such as an enhancer, a promoter, or a polyadenylation signal. “Gene expression cassette,” as defined herein, is a region of genetic material that has a sequence motif that regulates transcription, “Enhancer,” as defined herein, is a region of genetic material having a sequence motif that regulates transcription by increasing the likelihood that transcription will occur. “Promoter,” as defined herein, is a region of genetic material having a sequence motif that regulates transcription by binding to proteins involved in the initiation of transcription. “Polyadenylation signal,” as defined herein, regulates the addition of a poly(A) tail to mRNA.

In certain embodiments, the immunization is performed substantially free of vaccine additive. “Vaccine additive,” as defined herein, are ingredients included in an inoculum to enhance subject immunity, ensure vaccine safety and activity, and facilitate the vaccine's production. Vaccine additives may include preservatives such as Thimerosol, adjuvants such as aluminum salts, stabilizers such as sugars or gelatin, cell culture material such as egg protein, or inactivating ingredients such as formaldehyde.

In certain embodiments, the immunization is performed without using any adjuvant. In certain embodiments, material used for the immunization is substantially free from exogenous peptides and protein. “Substantially free from” means that any peptides or proteins are present only in inconsequential amounts, that is, in amounts that are not expected to have any effect on the generation of the animal's immune response. In certain embodiments, the animal is immunized using tail vein injection. In certain embodiments, the animal is immunized using intramuscular injection. In certain embodiments, the animal is immunized using intravascular injection via a vein other than the tail vein or via an artery. In certain embodiments, the animal is immunized using subcutaneous injection. In certain embodiments, the animal is immunized using intraperitoneal injection. In certain embodiments, the animal is immunized via injection into cerebrospinal fluid. In certain embodiments, the animal is immunized via retrograde intraductal injection. In certain embodiments, the animal is immunized by direct intraparenchymal injection.

The present disclosure also provides antibodies that recognize assembled viral capsids. Such antibodies may or may not have neutralizing ability. Neutralizing ability may be determined using methods known in the art. In certain embodiments, the antibodies are specific for a particular viral serotype, variant, or mutant, and do not substantially cross-react with other viral serotypes, variants, or mutants. The present disclosure also provides kits comprising such antibodies that may be used, for example, for ELISA, immunoblot, or immunofluorescence microscopy.

### EXAMPLES

The following examples are for illustration only. In light of this disclosure, those of skill in the art will recognize that variations of these examples and other embodiments of the disclosed subject matter are enabled without undue experimentation.

### Example 1—In Vivo AAV Viral Capsid Assembly in Mice by Plasmid DNA Transfection

AAV vector transduction mechanisms and the mechanisms of AAV capsid assembly were studied, including the roles and functions of AAP (assembly activating protein) in AAV virion formation. The present disclosure is based, in part, on the novel conception that AAV capsid assembly can take place in the mouse liver when AAV VP and AAP proteins are co-expressed in hepatocytes by DNA transfection and that assembled capsids can be secreted into the bloodstream and effectively immunize mice without infecting the mice with AAV viral particles. It was hypothesized that in vivo production of assembled capsids in the liver and their secretion into the bloodstream for AAP-independent serotypes (e.g., AAV4, AAV5 and AAV11) did not require co-expression of AAPs.

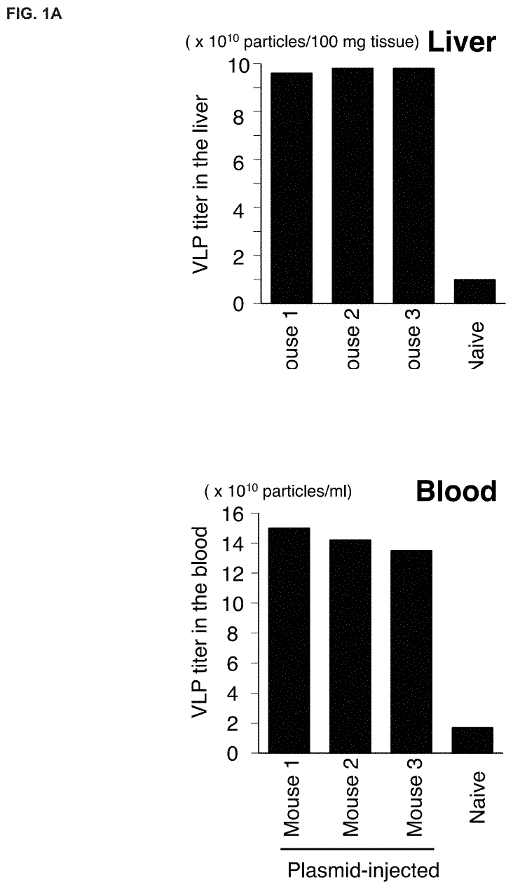

To investigate, 3 sixteen-week-old male C57BL/6J mice were injected via the tail vein using a hydrodynamic injection technique (e.g., HTV, which stands for Hydrodynamic Tail Vein injection) with a mixture of 50 μg of pCMV-AAV2VP3 and 50 μg of pCMV-AAP2 plasmids in a volume of 100 mL/kg saline. The injection was completed within less than 5 seconds (i.e., HTV). pCMV-AAV2VP3 and pCMV-AAP2 express AAV2 VP3 capsid protein and AAV2 AAP protein (i.e., AAP2) under the CMV-IE enhancer-promoter. There was one control animal that did not receive the treatment. Whole blood samples and the livers were collected three days post-injection. Using an AAV2 capsid-specific ELISA (AAV2 Titration ELISA, PROGEN), the concentrations of AAV2VP3 virus-like particles (VLPs) in the whole blood and the amount of VLPs in 100 mg liver tissue were determined. To prepare the liver tissue extract potentially containing VLPs, liver tissues were minced and incubated at 37° C. for 30 min in 140 mM NaCl, 5 mM KCl, 0.7 mM K₂HPO₄, 25 mM Tris-HCl, and 0.5% sodium deoxycholate according to the method reported by Walz et al, (Walz, Anisi et al. 1998). The liver extracts were adjusted to 200 mg liver tissue in 1 mL lysate. The data demonstrated that AAV2VP3 capsid assembly took place in the liver (as shown in FIG. 1A) and the resulting VLPs were secreted into the blood circulation (as shown in FIG. 1B). Assuming that the mouse body weight was 25 g, the blood volume was 7.2% of body weight, and that the liver weight was 1.35 g, the liver and the blood contained 1.3×10¹² particles and 2.6×10¹¹ particles, respectively. This indicated that by injecting 50 μg each of pCMV-AAV2VP3 and pCMV-AAP2 plasmid DNAs, a total of 1.6×10¹² particles could be produced in a mouse body, with the liver and the blood accounting for 84% and 16%, respectively.

Next, it was investigated whether the above-described method could trigger a significant immune response and produce antibodies against AAV2VP3 VLPs, which may or may not neutralize AAV2 infectivity. To this end, 5 ten-week-old BALB/cJ female mice were injected by HTV with a mixture of pCMV-AAV2VP3 and pCMV-AAP2 plasmids in the same manner as described above. The same injection was repeated at day 14 and day 21. Following the third plasmid injection, serum samples were collected and anti-AAV2 neutralizing antibodies (NAbs) were analyzed by our standard NAb assay (Adachi et al. Sci Rep 2020). In brief, following heat-inactivation of the serum samples at 56° C. for 30 min, the resulting serum samples were diluted with Dulbecco's Modified Eagle Medium (DMEM) at 1:10, 1:100, 1:1,000 and 1:10,000 ratios. Ten μL of each diluted serum sample and 10 μL of AAV2-CMV-luc (i.e., an AAV2 vector expressing the firefly luciferase gene product under the control of the CMV-IE enhance-promoter) preparation containing 1×10⁹ vg were incubated for 1 h at 37° C. After pretreatment with the wild-type adenovirus type 5, human embryonic kidney (HEK) 293 cells were treated with each test sample prepared as described above. Forty-eight hours later, the luciferase gene expression levels were quantified by the Bright-Glo™ Luciferase Assay kit (Promega). The neutralizing titer of the samples was determined as the highest dilution at which 50% or greater inhibition of the luciferase expression was observed.

As shown in Table 1, all five of the animals developed neutralizing antibodies against AAV2 following three plasmid injections. The titers ranged between dilutions of 1:1,000 and 1:10,000.

TABLE 1
AAV2 neutralizing antibodies developed in
mice injected with plasmid DNAs

| | Anti-AAV2 NAb titer |
|---|---|
| Mouse | (serum dilutions) |
| Mouse 1 (naïve) | <1:10 |
| Mouse 2 (naïve) | <1:10 |
| Mouse 3 (plasmid injected) | 1:1,000< and <1:10,000 |
| Mouse 4 (plasmid injected) | 1:1,000< and <1:10,000 |
| Mouse 5 (plasmid injected) | 1:1,000< and <1:10,000 |
| Mouse 6 (plasmid injected) | 1:1,000< and <1:10,000 |
| Mouse 7 (plasmid injected) | 1:1,000< and <1:10,000 |

### Example 2—Construction and Validation of Plasmids For Production of AAVAnc80 VLPs

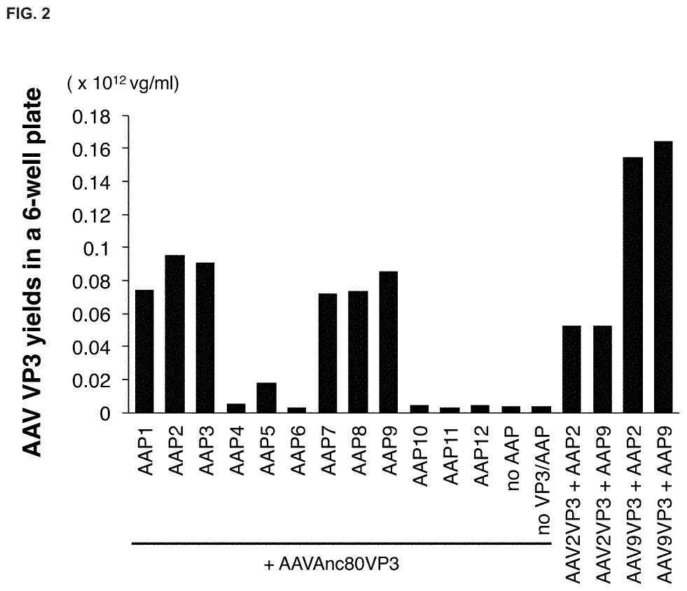

pCMV-AAVAnc80VP3 and pCMV-AAPAnc80 plasmids were constructed, and these plasmids were tested for AAVAnc80VP3 VLP production in HEK293 cells. It was found that AAVAnc80 VP3 only capsid can be assembled in HEK293 cells when AAV2 AAP (AAP2) or some of the other AAPs (AAP1, 3, 7, 8 and 9) are co-expressed, while the assembly did not take place in the absence of AAP (FIG. 2).

Although AAPAnc80 and other AAPs can also support AAVAnc80 VP3 capsid assembly, it was found that AAP2 supplementation results in higher VLP production than AAPAnc80 supplementation (data not shown). Based on this, the combination of pCMV-AAVAnc80VP3 and pCMV-AAP2 were used for DNA vaccination to produce antibodies against AAVAnc80 capsids.

Example 3—DNA Vaccination to Immunize Mice With Assembled AAVAnc80 Viral Capsids

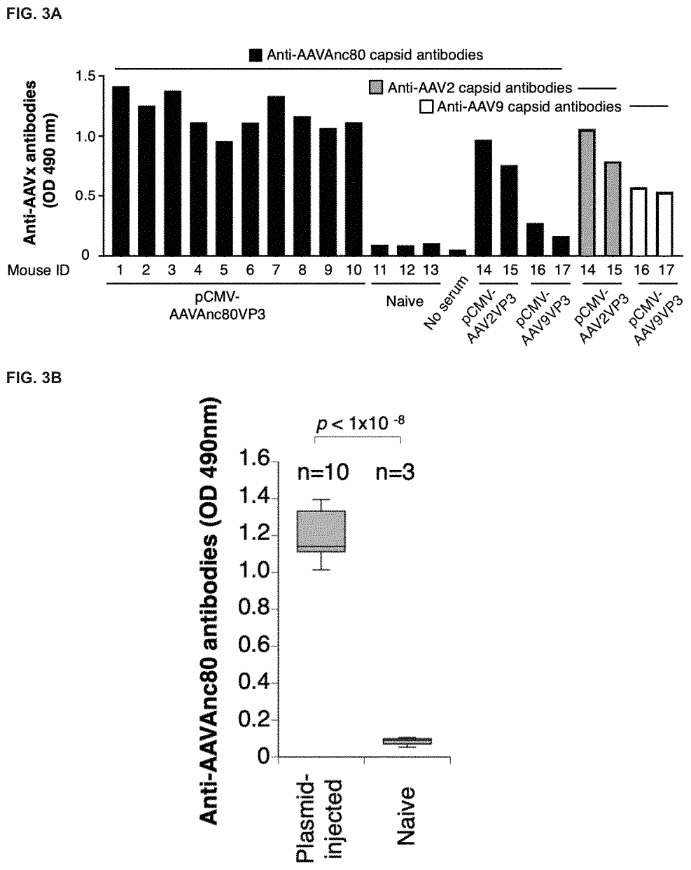

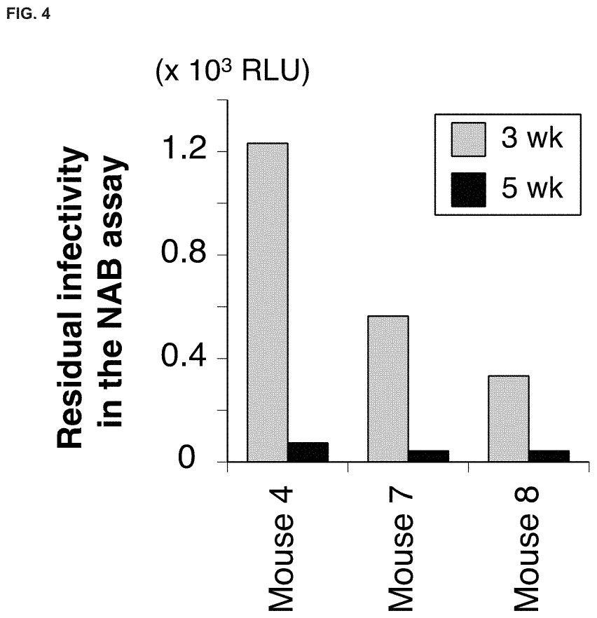

pCMV-AAVAnc80VP3 and pCMV-AAP2 plasmid DNAs were grown in E. coli using the standard method, extracted and purified by a commercially available plasmid extraction kit. The plasmids were then 0.45-μm filtered. To immunize mice with AAVAnc80 viral capsids, 10 nine-week-old BALB/cJ female mice were injected with pCMV-AAVAnc80VP3 and pCMV-AAP2 plasmid DNAs (50 μg each in 100 mL/kg of saline) via the tail vein using a hydrodynamic injection technique at days 0, 14, 21, 28 and 35, similar to the method reported by Bates et al. (Bates, Zhang et al. 2006). Three untreated animals were also included as controls. At the three-week time point after the third plasmid injection, serum samples were collected and anti-AAVAnc80 antibody titers were determined by an AAVAnc80 capsid antibody ELISA and a HEK293-based NAb assay using an AAVAnc80-CMV-luc vector. For the AAVAnc80 capsid antibody ELISA, Nunc MaxiSorp™ 96 well ELISA plates were coated with 1×10⁹ vg of AAVAnc80 vector per well (either AAVAnc80-CMV-luc or AAVAnc80-CMV-GFP). Serum samples were diluted at 1:10 with an ELISA dilution buffer and used for ELISA in duplicate. The results demonstrated that all 10 animals developed ELISA-positive antibodies against AAVAnc80 capsids (FIG. 3A). The ELISA optical density (OD) values were significantly higher than those of the untreated control animals with p<1×10⁻⁸ (FIG. 3B). All of these antibodies demonstrated neutralizing activity (Table 2). Following three injections of plasmid DNAs, NAb titers were in a range beyond the 1:1,000 serum dilution, and 9 out of 10 mice showed a >1:10,000 titer. Three animals were analyzed for the NAb titers again at the five-week time point after the completion of a full set of the immunization regimen (five injections of plasmid DNAs). A comparison of the relative light units (RLUs) in the NAb assays with AAVAnc80-CMV-luc vector between the 3-week time point and 5-week time point clearly demonstrates that the 5-week time point samples have higher NAb titers, although statistical approaches do not have the power to show a significance with p<0.05 (Welch's t-test) (FIG. 4).

In the ELISA shown in FIG. 3A, serum samples were included that were collected from two mice immunized by HTV injection of a mixture of pCMV-AAV2VP3 and pCMV-AAP2, and from two mice immunized by HTV injection of a mixture of pCMV-AAV9VP3 and pCMV-AAP9. These serum samples contained antibodies that reacted with not only corresponding AAV serotype capsids but also heterologous AAVAnc80 capsids due to cross-reactivity (FIG. 3A).

Table 2 represents the neutralizing antibody titer of the samples, which was determined as the highest dilution at which 50% or greater inhibition of the luciferase expression was observed.

TABLE 2
AAVAnc80 neutralizing antibodies developed
in mice injected with plasmid DNAs

| | Anti-AAVAnc80 | Anti-AAVAnc80 |
|---|---|---|
| | NAb titer at 3 wk | NAb titer at 5 wk |
| Mouse | (serum dilutions) | (serum dilutions) |
| Mouse 1 (naïve) | <1:100 | |
| Mouse 2 (naïve) | <1:100 | |
| Mouse 3 (naïve) | <1:100 | |
| Mouse 4 (plasmid injected) | >1:10,000 | >1:10,000 |
| Mouse 5 (plasmid injected) | >1:10,000 | |
| Mouse 6 (plasmid injected) | >1:10,000 | |
| Mouse 7 (plasmid injected) | >1:10,000 | >1:10,000 |
| Mouse 8 (plasmid injected) | >1:10,000 | >1:10,000 |
| Mouse 9 (plasmid injected) | 1:1,000< and <1:10,000 | |
| Mouse 10 (plasmid injected) | >1:10,000 | |
| Mouse 11 (plasmid injected) | >1:10,000 | |
| Mouse 12 (plasmid injected) | >1:10,000 | |
| Mouse 13 (plasmid injected) | >1:10,000 | |

### Example 4—Generation of Monoclonal Antibody-Producing Hybridoma Cell Clones

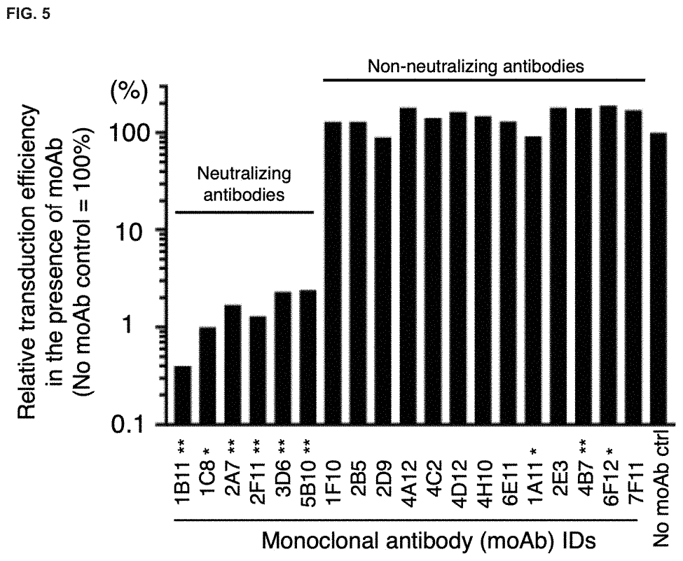

At the 6-week time point, 2 mice showing high AAVAnc80 NAb titers were selected for generation of monoclonal antibody-producing hybridoma cell lines. The spleens harvested from these two mice were sent to the Monoclonal Antibody Core at Oregon Health & Science University (OHSU). Splenocytes were then isolated and fused with a mouse myeloma cell line using a standard method. Approximately 700 hybridoma clones were screened for antibody production by an AAVAnc80 capsid antibody ELISA and AAVAnc80 capsid monomer antibody ELISA, resulting in the identification of a total of 19 clones producing monoclonal antibodies (FIG. 5). For the AAVAnc80 VP monomer ELISA, AAVAnc80 capsids were heat-denatured at 100° C. for 10 min in an ELISA coating buffer. None of the 19 clones reacted only with AAVAnc80 VP monomers (as shown in Table 3). All the 19 clones were cryopreserved.

### Example 5—Mouse Monoclonal Antibodies Against AAVAnc80 Capsids Were Categorized Into Three Distinct Groups

Through further characterization of the 19 monoclonal antibodies, including a cell-based NAb assay, it was found that the monoclonal antibodies may be categorized into the following three distinct groups: (Category 1) Monoclonal antibodies that can recognize only assembled capsids and do not react with capsid monomers, and have the ability to neutralize viral infectivity (6 clones; 1B11, 1C8, 2A7, 2F11, 3D6, and 5B10); (Category 2) Monoclonal antibodies that recognize only assembled capsids and do not react with capsid monomers, and do not have the ability to neutralize viral infectivity (8 clones; 1F10, 2B5, 2D9, 4A12, 4C2, 4D12, 4H10, and 6E11); and (Category 3) Monoclonal antibodies that recognize both assembled capsids and capsid monomers, and do not have the ability to neutralize viral infectivity (5 clones; 1A11, 2E3, 4B7, 6F12, and 7F11) (FIG. 5 and Table 3).

### Example 6—Immunoglobulin Classes, Isotypes and Light Chains of Anti-AAVAnc80 Capsid Mouse Monoclonal Antibodies

Immunoglobulin classes, isotypes, and light chains of nine monoclonal antibodies were determined (as shown in Table 3). Four of the monoclonal antibodies showed IgG1-κ, one showed IgG2a-κ, one showed IgG3-κ, one showed IgA-κ, and two showed IgM-κ. Anti-AAVAnc80 capsid monoclonal antibodies of IgA and IgM classes all showed weak binding to the antigen. The IgM class monoclonal antibodies showed cross reactivity to other serotypes. The IgG1 isotype was most commonly found among the identified monoclonal antibodies and all of them had the ability to neutralize infectivity. One monoclonal antibody that bound both assembled capsid and capsid monomer was an IgG2a antibody.

TABLE 3
A summary of anti-AAVAnc80 mouse monoclonal antibodies

| | | | Strength of | Class, | | | |
|---|---|---|---|---|---|---|---|
| | | | antigen | isotype, | Cross reactivity | | |
| Clone ID | Category | NAb? | binding | chain | AAV2 | AAV5 | AAV9 |
| 1B11 | 1 (Capsid only) | Yes | Strong | IgG1-κ | No | No | No |
| 1C8 | 1 (Capsid only) | Yes | Strong | IgG3-κ | No | No | No |
| 2A7 | 1 (Capsid only) | Yes | Strong | IgG1-κ | No | No | No |
| 2F11 | 1 (Capsid only) | Yes | Strong | IgG1-κ | Yes | No | No |
| 3D6 | 1 (Capsid only) | Yes | Strong | IgG1-κ | No | No | No |
| 5B10 | 1 (Capsid only) | Yes | Weak | IgA-κ | No | No | No |
| 4B7 | 3 (Capsid + | No | Weak | IgG2a-κ | No | No | No |
| | Monomer) | | | | | | |
| 1A11 | 3 (Capsid + | No | Weak | IgM-κ | Yes | Yes | Yes |
| | Monomer) | | | | | | |
| 6F12 | 3 (Capsid + | No | Weak | IgM-κ | Yes | Yes | Yes |
| | Monomer) | | | | | | |

### Example 7—Purification of Anti-AAVAnc80 Capsid Mouse Monoclonal Antibodies by Affinity Chromatography

To purify 6 select monoclonal antibodies from hybridoma culture media, each corresponding hybridoma cell line was grown in 200 mL culture. Affinity chromatography purification of monoclonal antibodies was carried out at the OHSU Monoclonal Antibody Core using Thermo Scientific™ Pierce™ Protein G Agarose and according to the Core's standard purification protocol. Table 4 summarizes the concentration of immunoglobulins in each purified monoclonal antibody reagent.

TABLE 4
Concentrations of affinity-purified anti-AAVAnv80
capsid mouse monoclonal antibodies

| moAb | | Concentration (mg/mL) |
|---|---|---|
| 2F11 | (IgG1-κ) | 5.0 |
| 3D6 | (IgG1-κ) | 3.6 |
| 5B10 | (IgA-κ) | 1.7 |
| 4B7 | (IgG2a-κ) | 3.2 |
| 1B11 | (IgG1-κ) | 3.1 |
| 2A7 | (IgG1-κ) | 2.6 |

### Example 8—Anti-AAVAnv80 Capsid Mouse Monoclonal Antibodies 1B11, 2A7, 4B7 and 3D6 Were AAVAnc80 Capsid Specific and Did Not Cross React With AAV2 Capsid

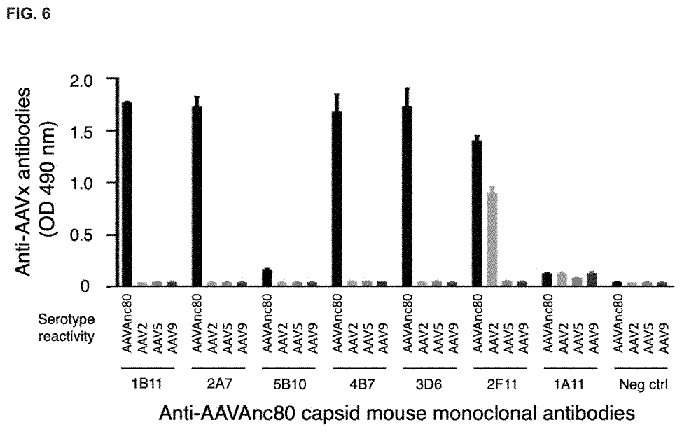

Cross-reactivity of anti-AAVAnc80 capsid monoclonal antibodies was tested by anti-AAVx capsid antibody ELISAs (x=Anc80, 2, 5, and 9). Among 9 monoclonal antibodies tested, the two IgM monoclonal antibodies cross-reacted with AAV2, AAV5, and AAV9 capsids (Table 3). One IgG1 monoclonal antibody (2F11) cross-reacted with AAV2 capsid but did not bind to AAV5 or AAV9 capsids. In contrast, monoclonal antibodies 1B11, 2A7, 4B7, and 3D6 only reacted with AAVAnc80 capsids among the four AAV capsids tested (as shown in FIG. 6). Although it was unknown whether 1B11, 2A7, 4B7, and 3D6 could react with AAV serotypes other than AAV2, 5, and 9, this data demonstrated high antigen specificity of the monoclonal antibodies 1B11, 2A7, 4B7, and 3D6. Based on the information available at the time, this was the first description of antibodies that are highly specific to AAVAnc80 capsids that do not react with other major common AAV serotypes.

### Example 9—Affinity-Purified Anti-AAVAnv80 Capsid Mouse Monoclonal Antibodies, 2A7 and 1B11 Had a Significant Neutralizing Ability

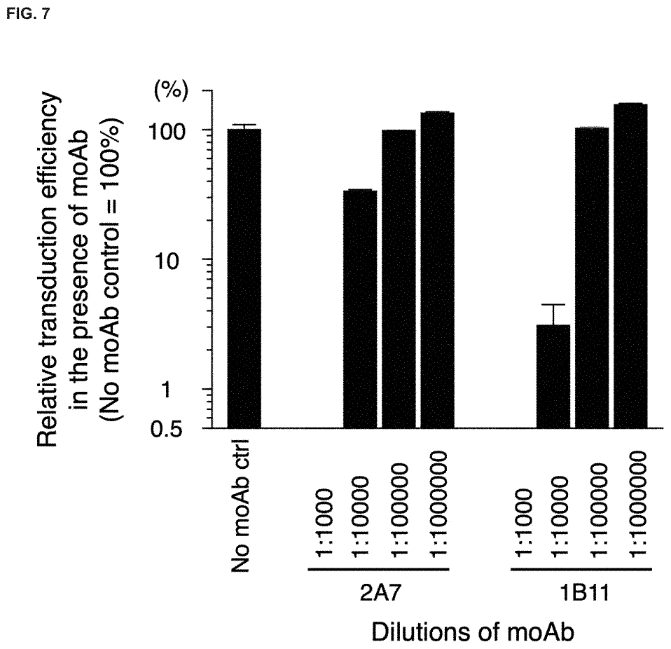

Affinity-purified anti-AAVAnc80 capsid monoclonal antibodies 2A7 (2.6 mg/mL) and 1B11 (3.1 mg/ML) were diluted by 1,000, 10,000, 100,000, and 1,000,000-fold. Ten μL of the diluted moAb samples were reacted with 10⁹ vg of AAVAnc80-CMV-luc in a 20 μL reaction volume and incubated at 37° C. for 1 h. Meanwhile, 2×10⁴ CHO-K1 cells/well in a 96-well plate were infected with human adenovirus type 5 and incubated at 37oC for 1 h. The moAb/AAV mixtures were then added to the cells together with media to make a total of 200 μL/well. Cells were incubated two days to allow for AAVAnc80-CMV-luc infection and expression of luciferase. Luciferase expression levels were then determined by Bright-Glo Luciferase Assay detection kit (Promega) and expressed as relative light units (RLUs). RLU values were used to determine relative transduction efficiencies of tested conditions, which are values normalized with the RLU values obtained from the AAV only (no moAb) controls. NAb titer was defined as the highest dilution that suppressed the relative transduction efficiency by >50%. With this definition, the affinity-purified moAbs, 2A7 and 1 B11, had NAb titers of >1:10,000 (FIG. 7).

### Example 10—Establishment of AAVAnc80 Capsid Titration ELISA Kits

With the purified and characterized anti-AAVAnc80 capsid monoclonal antibodies 2A7 and 1B11 in hand, it was sought to establish AAVAnc80 capsid titration ELISA kits. AAVAnc80 capsid can be recognized by anti-AAV2 capsid mouse monoclonal antibody A20 and anti-AAV8 capsid mouse monoclonal antibody ADK8 via these monoclonal antibodies' non-specific cross-reactivity across different serotypes. A20 and ADK8 are commercially available through several vendors including PROGEN. Although AAVAnc80 capsid titration by ELISA might be possible using these cross-reacting non-AAVAnc80 mouse monoclonal antibodies, no AAVAnc80 capsid titration ELISA kits existed that used anti-AAVAnc80 capsid-specific monoclonal antibodies. To establish anti-AAVAnc80 capsid moAb-based sandwich ELISAs for AAVAnc80 capsid titration, biotinylated 2A7 and 1B11 moAbs were first generated. To this end, purified 2A7 and 1B11 moAbs were dialyzed against phosphate-buffered saline (PBS) without calcium and magnesium (PBS(−)) to remove sodium azide (NaN₃) contained in the moAb stocks. Each NaN₃-free moAb was biotinylated by spontaneous reaction of its primary amines with Biotin-7-NHS, using a commercially available kit (Biotin Protein Labeling Kit, Millipore Sigma). The manufacturer's instructions for reaction incubation and column chromatography to elute biotinylated moAb were followed. Eluted protein concentration was measured by absorbance on a BioTek Epoch, using the “IgG” function. After pooling the top biotinylated protein-containing fractions, 1% bovine serum albumin (BSA) was added to increase the overall protein concentration and prevent loss of moAb due to nonspecific binding to tube walls, according to the manufacturer's recommendation. 0.05% NaN₃ was then added to prevent growth of contaminants during storage at 4° C.

Sandwich ELISAs to quantify AAVAnc80 particles were then developed. In brief, an anti-AAVAnc80 moAb (either 2A7 or 1B11) was coated onto the bottom of MaxiSorp™ Immuno Clear Standard Modules in a 0.1M carbonate-bicarbonate buffer and incubated overnight at 4° C. (100 ng/well). After being washed, plates were blocked with the blocking/sample dilution buffer at 37° C. for 1 h. After the plates were washed, known amounts of AAVAnc80 particles in the blocking/sample dilution buffer were added to the wells and incubated at 37° C. for 1 h. After the plates were washed, biotinylated anti-AAVAnc80 moAb (either 2A7 or 1B11) diluted in the blocking/sample dilution buffer was added to the wells and incubated at 37° C. for 1 h. After the plates were washed, Pierce™ High Sensitivity Streptavidin-HRP was added and incubated at 37° C. for 1 h. Finally, a chromogenic substrate OPD was added and OD490 was determined for each well. Amounts and dilutions of primary moAb antibodies, biotinylated moAbs and Streptavidin-HRP, and type of the blocking/sample dilution buffer were experimentally optimized.

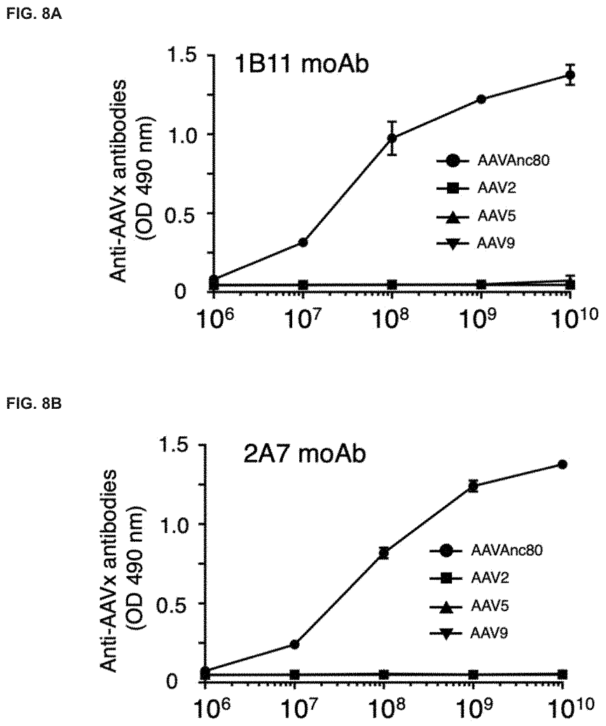

Using AAVAnc80 vector capsids purified by two cycles of CsCl gradient ultracentrifugation that could remove the majority of empty capsids, the sensitivity of the AAVAnc80 capsid titration of the developed ELISAs was determined. The ELISAs could detect at least 1×10⁶ vg of AAVAnc80 vectors in 50 μL per well. OD490 values did not reach saturation even at 1×10¹⁰ vg of AAVAnc80 vectors in 50 μL per well, showing a 4-log dynamic range (FIG. 8).

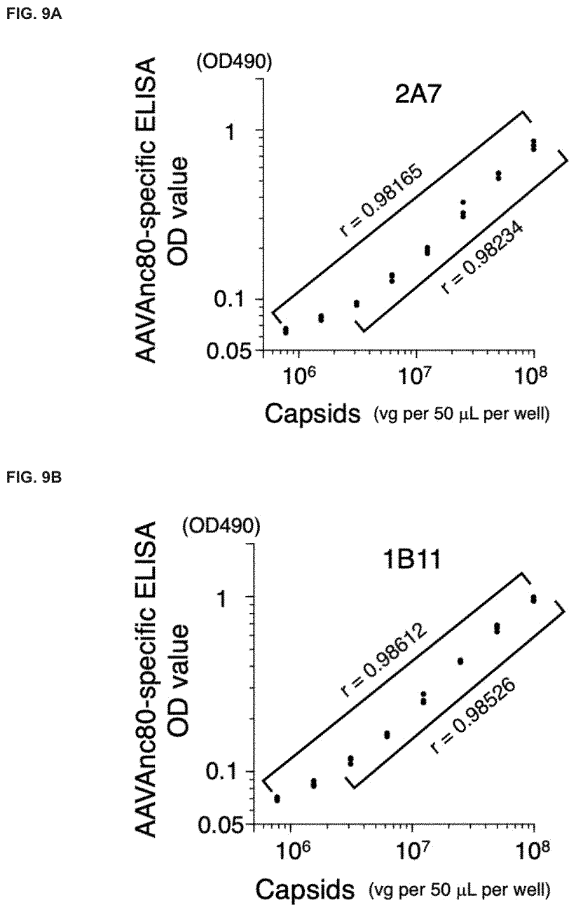

Using AAVAnc80 capsid standards prepared by a 2-fold serial dilution in a range between 7.8×10⁵ and 1.0×10⁸ vg per 50 μL per well, standard curves were drawn to determine the linear dynamic range of the ELISA to be used for capsid quantification. This assay revealed that an unambiguous linear range is observed between 3.2×10⁶ and 1×10⁸ vg in 50 μL per well with Pearson's correlation coefficient r=0.98234 for moAb 2A7 and r=0.98526 for moAb 1B11 (FIG. 9). Although OD490 values for 7.8×10⁵ and 1.6×10⁶ vg are not on the regression lines, they did not significantly deviate from the regression line (as shown in FIG. 9). By using an alternative regression model such as four parameter logistic regression, it was possible to expand the quantitative dynamic range of the AAVAnc80 capsid titration ELISAs from 1×10⁸ vg per 50 μL down to 7.8×10⁵.

### REFERENCES CITED

All references cited in this disclosure are incorporated by reference in their entirety.
Adachi, K., et al. (2020). “Adeno-associated virus-binding antibodies detected in cats living in the northeastern United States lack neutralizing activity.” Sci Rep 10(1): 10073.
Bates, M. K., et al. (2006). “Genetic immunization for antibody generation in research animals by intravenous delivery of plasmid DNA.” Biotechniques 40(2): 199-208.
Earley, L. F., et al. (2015). “Identification and characterization of nuclear and nucleolar localization signals in the adeno-associated virus serotype 2 assembly-activating protein.” J Virol 89(6): 3038-3048.
Earley, L. F., et al. (2017). “Adeno-associated Virus (AAV) Assembly-Activating Protein Is Not an Essential Requirement for Capsid Assembly of AAV Serotypes 4, 5, and 11.” J Virol 91(3): e01980-16
Liu F. et al., (1999). “Hydrodynamics-Based Transfection in Animals by System Administration of Plasmid DNA.” Gene Therapy 6; 1258-1266.
Powers, J. M., et al. (2018). “A Quantitative Dot Blot Assay for AAV Titration and Its Use for Functional Assessment of the Adeno-associated Virus Assembly-activating Proteins.” J Vis Exp (136): e56766.
Suda T and Liu D., (2007) “Hydrodynamic gene delivery: its principles and applications.” Molecular Therapy 15(12): 2063-69.
Walz, C. M., et al. (1998). “Detection of infectious adeno-associated virus particles in human cervical biopsies.” Virology 247(1): 97-105.

It will be obvious to those having skill in the art that many changes may be made to the details of the above-described embodiments without departing from the underlying principles of the invention. The scope of the present invention should, therefore, be determined only by the following claims.

1. A method of producing, in vivo, antibodies against viral capsids (VCs) derived from a non-enveloped virus (NEV), the method comprising the steps of:
(1) administering, to a subject, by hydrodynamic-based transfection, a first set of genetic material encoding one or more NEV structural proteins for inducing the subject's intracellular translation and assembly of the NEV structural proteins into one or more VCs thereby generating, by the subject, antibody-producing cells that secrete anti-VC antibodies;
(2) (optionally) repeating step 1.

2. The method of claim 1, further comprising administering, to the subject, by hydrodynamic-based transfection, a second set of genetic material encoding one or more NEV non-structural proteins for facilitating the subject's intracellular assembly of the NEV structural proteins into one or more VCs.

3. The method of claim 1 or 2, further comprising harvesting the antibody-producing cells and/or anti-VC antibodies from the subject.

4. The method of claim 1, 2, or 3, further comprising:
generating a plurality of hybridomas from the antibody-producing cells;
screening the plurality of hybridomas and selecting one or more hybridomas within the plurality for culture;
culturing one or more of the selected hybridomas.

5. The method of claim 1, in which step 1 is repeated 1 to 10 times.

6. The method of claim 5, in which the time intervals between repeating step 1 are about 1 day to about 28 days.

7. The method of claim 1, 2, or 3, in which the first and second set of genetic material are at least one nucleic acid molecule chosen from a ssDNA, a dsDNA, a linear pDNA, a circular pDNA, a ssRNA, a dsRNA, a circRNA, a mRNA, a microRNA, an siRNA, and a sgRNA.

8. The method of claim 1, 2, or 3, in which the first set of genetic material encodes at least one protein chosen from a NEV-based VP capsid protein, an AAV VP capsid protein, an AAV2 VP capsid protein, and an AAVAnc80 VP capsid protein.

9. The method of claim 1, 2, or 3, in which the second set of genetic material encodes at least one protein chosen from a NEV-based non-structural protein, an AAV-based AAP, an AAV2 AAP, and an AAPAnc80.

10. The method of claim 1, 2, or 3, in which either the first or second set of genetic material encode a gene expression cassette chosen from an enhancer, a promoter, and a polyadenylation signal.

11. The method of any of claims 1 to 4, in which the non-enveloped virus is classified within the family Parvoviridae, or is an adeno-associated virus (AAV), a synthetic AAV, or an AAV vector.

12. The method of claim 11, in which the AAV vector is an AAVAnc80 vector, an AAV2 vector, or an AAV9 vector.

13. The method of any of claims 1 to 4, in which the hydrodynamic-based transfection is performed substantially free of vaccine additive.

14. The method of any of claims 1 to 4, in which the subject is an animal.

15. The method of claim 14, in which the animal is a rodent.

16. The method of claim 15, in which the rodent is a mouse.

17. The method of any of claims 1 to 4, in which the anti-VC antibodies substantially neutralize a NEV's infectivity.

18. The method of any of claims 1 to 4, in which the anti-VC antibodies do not substantially neutralize a NEV's infectivity.

19. The method of any of claims 1 to 4, in which the anti-VC antibodies do not substantially cross-react with other viral serotypes.

20. The method of any of claims 1 to 4, in which the VCs constitute one or more substantially assembled, or partially assembled, NEV viral capsids.

21. The method of any of claims 1 to 4, in which the anti-VC antibodies are mouse monoclonal antibodies.
